(19) United States
(12) Patent Application Publication
(10) Pub. No.: US 2025/0271154 A1
(43) Pub. Date: Aug. 28, 2025

(54) CABINET DEHYDRATOR

(71) Applicant: DISH Wireless L.L.C., Englewood, CO (US)

(72) Inventors: Warren Newell Bloss, III, Castle Rock, CO (US); Salim Fermin, Castle Rock, CO (US); Terry Andrew Thompson, Marietta, GA (US)

(21) Appl. No.: 18/589,082

(22) Filed: Feb. 27, 2024

(51) Int. Cl. F24F 3/14; G01N 33/00; G01N 33/18; H05K 7/14; H05K 7/20

(52) U.S. Cl. CPC F24F 3/1411; G01N 33/0027; G01N 33/18; H05K 7/1488; H05K 7/20172; H05K 7/20209

(57) ABSTRACT

A dehydrator unit comprises an enclosed housing that houses one or more removable desiccant cartridges designed to remove moisture from air, a front panel comprising one or more front fans that cause air to flow into the enclosed housing, a back panel comprising one or more back fans that cause air to flow out of the enclosed housing, and a humidity sensor that measures humidity in air surrounding the dehydrator unit. A controller receives a humidity value measured by the humidity sensor and compares the humidity value to a humidity threshold. If the humidity value exceeds the humidity threshold, the controller turns on the one or more front fans, the one or more back fans, or a combination thereof.

### TECHNICAL FIELD

The present disclosure relates generally to controlling moisture in an enclosed space, and more specifically to a cabinet dehydrator for regulating moisture inside an enclosed cabinet.

### BACKGROUND

A network rack, also known as a network equipment rack or network cabinet, is a specialized type of rack or cabinet designed to house and organize electronic networking equipment and related hardware in data centers, server rooms, network closets, or telecommunications facilities. Network racks serve a specific purpose, which is to provide a structured and secure environment for networking components, such as switches, routers, patch panels, modems, and other networking devices. To avoid malfunction and/or failure of electronic equipment deployed inside a network cabinet it is important that a low moisture environment is maintained inside the network cabinet.

### SUMMARY

The system and methods implemented by the system as disclosed in the present disclosure provide an intelligent technique to regulate moisture/humidity inside a network cabinet.

The disclosed system and methods provide several practical applications and technical advantages. For example, the disclosed system and method provide the practical application of maintaining low moisture/humidity inside a network cabinet. As described in embodiments of the present disclosure, a dehydrator unit is designed and configured to regulate moisture levels inside a network cabinet so that low moisture levels are maintained inside the network cabinet. The dehydrator unit includes an enclosed housing, a front panel disposed at a front end of the enclosed housing, and a back panel disposed at a back end of the enclosed housing. The front panel may include one or more fans that cause air to flow into the enclosed housing. The back panel may include one or more fans that expel air out of the enclosed housing. The dehydrator unit further includes a desiccant tray that is configured to be housed inside the enclosed housing and hold one or more desiccant cartridges on a top surface of the desiccant tray. Each desiccant cartridge includes a desiccant material that removes moisture from ambient air. The dehydrator unit further includes a humidity sensor that is configured to measure humidity levels in the air surrounding the dehydrator unit. The dehydrator unit additionally includes a controller that controls operation of the dehydrator unit. The controller receives humidity measurements from the humidity sensor and compares the humidity measurements to a pre-set humidity threshold. When one or more humidity measurements exceed the humidity threshold, the controller turns on one or more front fans, one or more back fans, or a combination thereof. Turning on the front fans and/or back fans allows air flow across the desiccant cartridges, which in turn allows the desiccant cartridges to remove moisture from the air flowing across the desiccant cartridges. The controller continues to receive and compare humidity measurements to the humidity threshold. When one or more humidity measurements are found to be equal or lower than the humidity threshold, the controller turns off the one or more front fans, the one or more back fans, or the combination thereof that were previously turned on. Thus, dehydrator unit maintains a low moisture environment inside the network cabinet.

By maintaining a low moisture environment inside the network cabinet, the disclosed dehydrator unit prevents malfunction, damage and/or failure of electronic equipment deployed inside the network cabinet, thus improving operation of the electronic equipment and avoiding any downtime resulting from malfunctioning and/or failed electronic equipment. Additionally, by avoiding malfunction and failure of network equipment housed in the network cabinets, the disclosed system and methods generally improve network performance of a wireless network (e.g., cellular network) in which the network equipment is deployed.

Thus, the disclosed system and methods generally improve the technology related to cellular networks and communication using such networks.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1A illustrates a front view of an example network cabinet;

FIG. 1B illustrates a back view of the example network shown in FIG. 1A;

FIG. 1C illustrates an example dehydrator unit deployed inside a network cabinet shown in FIGS. 1A and 1B, in accordance with one or more embodiments of the present disclosure;

FIG. 2 illustrates an example design of the dehydrator unit shown in FIG. 1C, in accordance with one or more embodiments of the present disclosure;

FIG. 3A illustrates an example perspective front view of the dehydrator unit shown in FIG. 2, in accordance with one or more embodiments of the present disclosure;

FIG. 3B illustrates an example perspective back view of the dehydrator unit shown in FIG. 2, in accordance with one or more embodiments of the present disclosure; and

FIG. 4 is a flowchart of an example method for regulating moisture in a network cabinet, in accordance with embodiments of the present disclosure.

### DETAILED DESCRIPTION

### System Overview

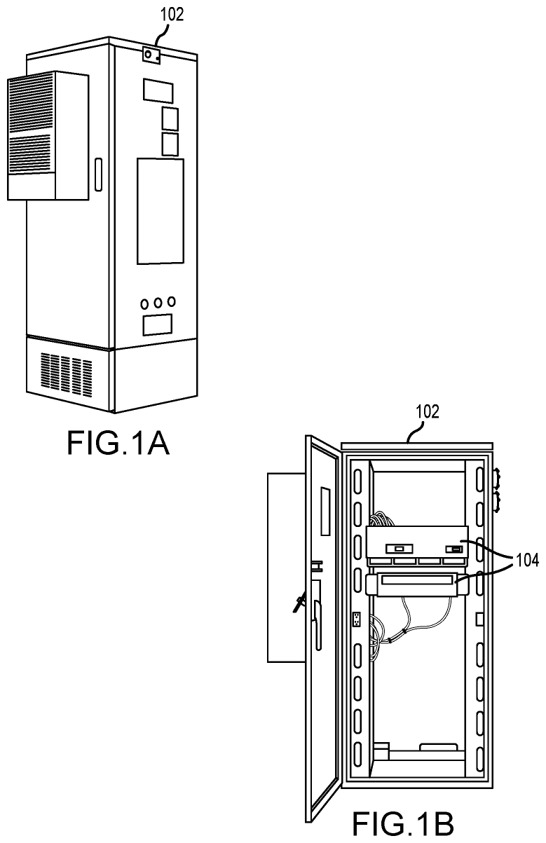

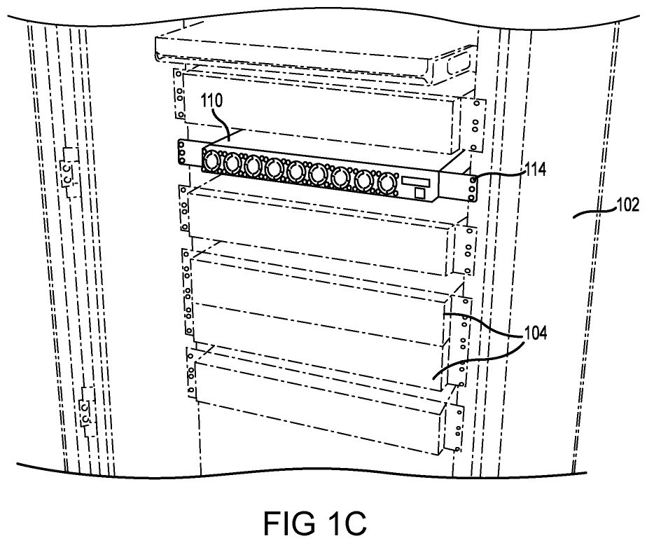

FIGS. 1A and 1B illustrate a front view and a back view respectively of an example network cabinet 102. A network cabinet 102 is a specialized type of enclosed rack designed to house and organize electronic equipment 104 and related hardware in data centers, server rooms, network closets, or telecommunications facilities. Network cabinets (e.g., network cabinet 102) serve a specific purpose, which is to provide a structured and secure environment for networking components, such as switches, routers, patch panels, modems, and other networking devices. For example, the network cabinet 102 may house electronic equipment 104 including, but not limited to one or more of a power supply, a router, a surge protector, a power booster, or a backup power supply having Lithium-ion (Li-ion) batteries. In one embodiment, a network cabinet 102 is deployed at a cell site of a wireless cellular network and houses various electronic network equipment supporting the cell site. High levels of moisture inside the network cabinet 102 may cause electronic equipment 104 to malfunction or fail. In some cases, a high moisture environment inside a network cabinet 102 may damage electronic equipment 104 stored inside the network cabinet 102. For example, condensation due to high moisture inside the network cabinet 102 may cause electric short circuit inside a piece of electronic equipment 104 and permanently damage the piece of electronic equipment 104. In another example, condensation may cause a catastrophic failure of a Li-ion battery due to a sudden release of energy in the form of heat from a short circuit, which may lead to loss of the entire network cabinet 102. Thus, to avoid malfunction and/or failure of electronic equipment 104 deployed inside a network cabinet 102, it is important that a low moisture environment is maintained inside the network cabinet 102.

Embodiments of the present disclosure discuss a dehydrator unit 110 that regulates moisture levels inside a network cabinet 102 so that low moisture levels are maintained inside the network cabinet 102. For example, FIG. 1C illustrates an example dehydrator unit 110 deployed inside a network cabinet 102. The design of the dehydrator unit 110 will be discussed in detail further below. As shown in FIG. 1C, the dehydrator unit 110 is designed to be installed at any vertical position inside the network cabinet 102 using screws 114. However, a person having ordinary skill in the art may appreciate that the dehydrator unit 110 may be configured for installation in other designs of the network cabinet 102.

It may be noted that while embodiments of the present disclosure discuss an example deployment of the dehydrator unit 110 in a network cabinet 102, a person having ordinary skill in the art may appreciate that the dehydrator unit 110 may be deployed to regulate moisture in any enclosed space.

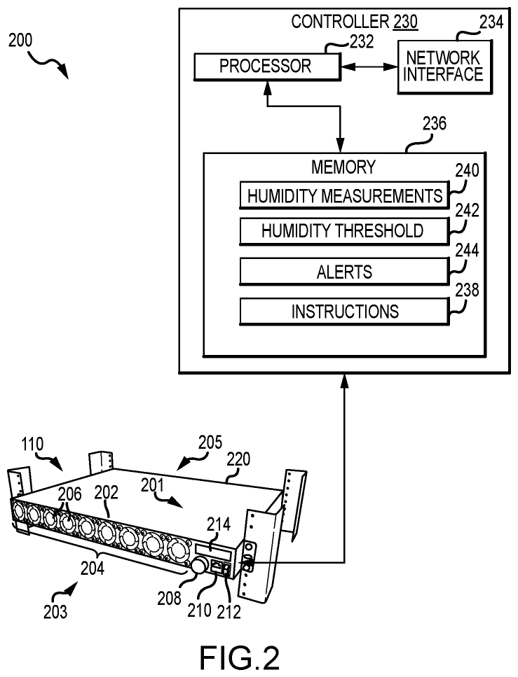

FIG. 2 illustrates an example design 200 of the dehydrator unit 110 shown in FIG. 1C, in accordance with one or more embodiments of the present disclosure. As shown in FIG. 2, the dehydrator unit 110 includes an enclosed housing 201, a front panel 202 disposed at a front end 203 of the enclosed housing 201, and a back panel 220 disposed at a back end 205 of the enclosed housing 201. In one embodiment, the dehydrator unit 110 is configured to be installed inside a network cabinet 102 such that the front end 203 of the dehydrator unit 110 is positioned adjacent to a face of the network cabinet 102 that can be opened to at least access the dehydrator unit 110.

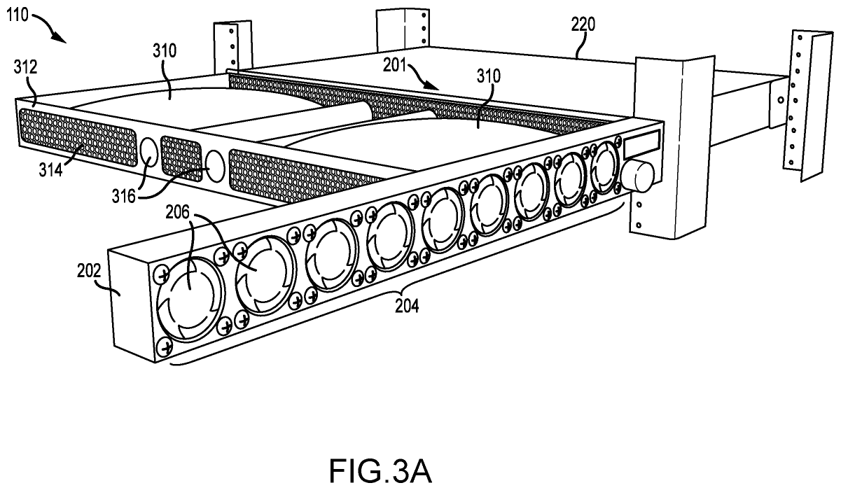

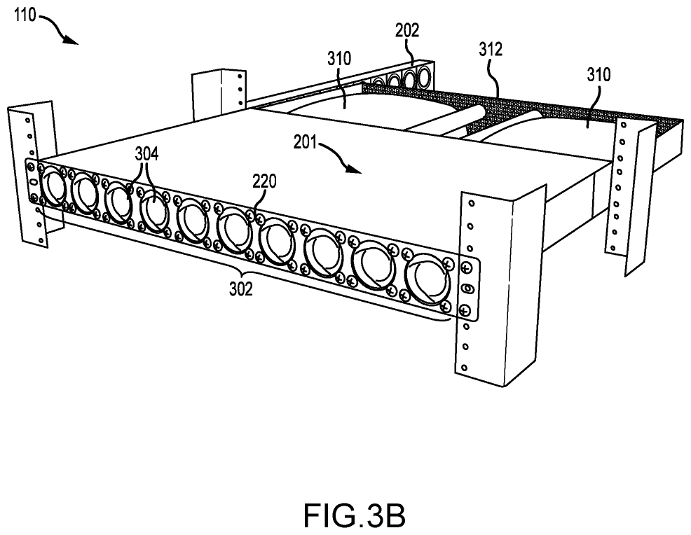

The enclosed housing 201 is configured to house one or more removable desiccant cartridges that remove moisture from air. FIG. 3A illustrates an example perspective front view of the dehydrator unit 110, in accordance with one or more embodiments of the present disclosure. As shown in FIG. 3A, the dehydrator unit 110 includes a desiccant tray 312 that is configured to be housed inside the enclosed housing 201 and hold one or more desiccant cartridges 310 on a top surface of the desiccant tray 312. In the embodiment shown in FIG. 3A, the desiccant tray 312 is slidably engaged with the enclosed housing 201 such that the desiccant tray 312 slides in and out of the enclosed housing 201. The front panel 202 may be configured to be opened to expose a front end of the desiccant tray 312. For example, as shown in FIG. 3A, the front panel 202 is configured to swing open and expose a front end of the desiccant tray 312. While FIG. 3A illustrates the front panel 202 configured to swing open on one side of the enclosed housing 201, a person having ordinary skill in the art may appreciate that other mechanisms to open the front panel 202 may be employed. For example, the front panel 202 may be hinged to swing open on the other side of the enclosed housing 201, hinged along the length of the enclosed housing 201 to swing open upwards or swing open downwards, and or designed to be removable from the enclosed housing 201. In any case, this mechanism allows a user to open the front panel 202 and slide out the desiccant tray 312 to gain access to the desiccant cartridges 310. In one embodiment, indentations and/or holes may be provided at the front end of the desiccant tray 312, wherein each of the one or more indentation/holes is configured to receive a finger of a user for holding the desiccant tray 312 and sliding out the desiccant tray 312 from the enclosed housing 201. For example, as shown in FIG. 3A, two holes/indentations 316 are built into the front end of the desiccant tray 312, wherein the holes 316 are spaced apart to allow a user to hold the desiccant tray 312 using a thumb and another finger of a user's hand. In one embodiment, as shown in FIG. 3A, at least the front end of the desiccant tray 312 facing the front panel 202 and a back end of the desiccant tray 312 facing the back panel 220 is made of a mesh material 314 to allow air flow across desiccant tray 312.

Each desiccant cartridge 310 includes a desiccant material that removes moisture from ambient air. For example, the desiccant material in the desiccant cartridges 310 may include silica gel that removes moisture from air through the process of adsorption. Silica gel is a desiccant made from silicon dioxide found in sand and quartz. It stands out from other desiccants due to its high moisture adsorption capacity, non-corrosive nature, non-toxicity, regenerative quality, and versatility in terms of size and shape. These qualities make it a preferred choice in various industries and applications where controlling humidity is crucial. Additionally, desiccant cartridges 310 made of/containing silica gel may be reusable. For example, once a desiccant cartridge 310 made of silica gel reaches maximum moisture saturation, the desiccant cartridge 310 may be reactivated with heat to dry out the moisture and may be re-used.

In one embodiment, the desiccant material in a desiccant cartridge 310 may be designed to chemically change color based on a level of moisture saturation. This means that a color of a desiccant cartridge 310 may indicate a level of moisture saturation in the desiccant cartridge 310. For example, the desiccant material used for the desiccant cartridges 310 includes colored silica gel that changes from a first color when moisture saturation of the silica gel is below a first saturation level to a second color when the moisture saturation of the silica gel exceeds a second saturation level. In one embodiment, blue silica gel may be used that has cobalt chloride, which is blue when dry but changes to pink color when it has reached its maximum or close to maximum adsorption capacity. In an alternative embodiment, orange silica gel may be used that has naturally occurring mineral silicon dioxide, which is orange when dry but changes to green color when it has reached its maximum or close to maximum adsorption capacity. It may be noted that the colored silica gel may go through intermediate shade transitions that indicate intermediate moisture saturation levels.

While silica gel is one example of desiccant material that can be used for the desiccant cartridges, a person having ordinary skill in the art may appreciate that any known desiccant material may be used in the desiccant cartridges 310. Further, while the example design of the dehydrator unit 110 shown in FIG. 3A illustrates two desiccant cartridges 310 housed inside the enclosed housing 201, a person having ordinary skill in the art may appreciate that any number of desiccant cartridges 310 of any shape and/or size may be housed inside the enclosed housing 201.

In one embodiment, the front panel 202 may include one or more fans that cause air to flow into the enclosed housing 201. For example, as shown in FIGS. 2 and 3A, the front panel 202 includes a front fan array 204 having a plurality of front fans 206 that are configured to force air into the enclosed housing 201. In an additional or alternative embodiment, the back panel 220 may include one or more fans that expel air out of the enclosed housing 201. For example, as shown in FIG. 3B which illustrates an example perspective back view of the dehydrator unit 110, the back panel 220 includes a back fan array 302 having a plurality of back fans 304 that expel air out of the enclosed housing 201. Thus, when the front fans 206 and the back fans 304 are running, a steady air flow is maintained across the desiccant tray 312 allowing the desiccant cartridges 310 to efficiently remove moisture from the air inside the network cabinet 102.

It may be noted that while FIGS. 2, 3A and 3B illustrate a specific design of the dehydrator unit 110 including both the front fan array 204 and the back fan array 302, the dehydrator unit 110 may include only one of the front fan array 204 or the back fan array 302. For example, the dehydrator unit 110 may include the front fan array 204 only and may include openings (e.g., holes) at the back end 205 of the enclosed housing 201 to allow air to leave the enclosed housing 201. Alternatively, the dehydrator unit 110 may include the back fan array 302 only and may include openings (e.g., holes) at the front end 203 of the enclosed housing 201 to allow air to enter the enclosed housing 201.

As shown in FIG. 2, the dehydrator unit 110 further includes a humidity sensor 208 that is configured to measure humidity level in the air surrounding the dehydrator unit 110. In other words, the humidity sensor 208 measures humidity levels inside the network cabinet 102. As further described below, to regulate humidity/moisture levels inside the network cabinet 102, the front fans 206 and/or back fans 304 may be turned on or turned off based on humidity values measured by the humidity sensor 208.

Referring to FIG. 2, the dehydrator unit 110 may further include a controller 230 that controls operation of the dehydrator unit 110. The controller 230 includes a processor 232, a memory 236, and a network interface 234. The controller 230 may be configured as shown in FIG. 2 or in any other suitable configuration.

The processor 232 may include one or more processors operably coupled to the memory 236. The processor 232 is any electronic circuitry including, but not limited to, state machines, one or more central processing unit (CPU) chips, logic units, cores (e.g., a multi-core processor), field-programmable gate array (FPGAs), application specific integrated circuits (ASICs), or digital signal processors (DSPs). The processor 232 may be a programmable logic device, a microcontroller, a microprocessor, or any suitable combination of the preceding. The processor 232 is communicatively coupled to and in signal communication with the memory 236. The one or more processors are configured to process data and may be implemented in hardware or software. For example, the processor 232 may be 8-bit, 16-bit, 32-bit, 64-bit or of any other suitable architecture. The processor 232 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components.

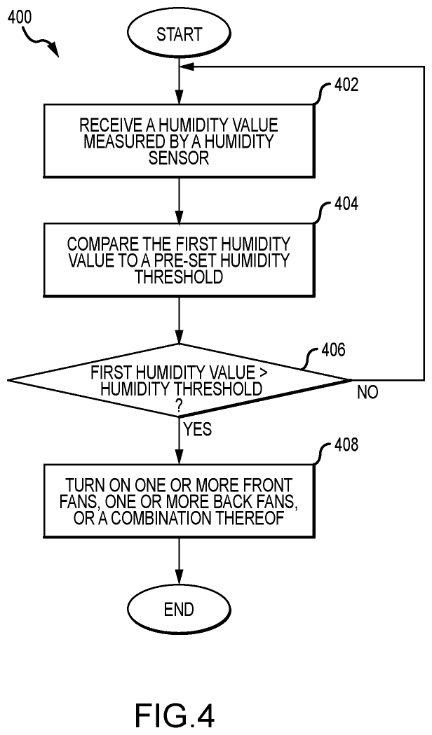

The one or more processors are configured to implement various instructions, such as software instructions. For example, the one or more processors are configured to execute instructions 238 to implement the controller 230. In this way, processor 232 may be a special-purpose computer designed to implement the functions disclosed herein. In one or more embodiments, the controller 230 is implemented using logic units, FPGAs, ASICs, DSPs, or any other suitable hardware. The controller 230 is configured to operate as described with reference to FIG. 4. For example, the processor 232 may be configured to perform at least a portion of the method 400 as described in FIG. 4.

The memory 236 comprises a non-transitory computer-readable medium such as one or more disks, tape drives, or solid-state drives, and may be used as an over-flow data storage device, to store programs when such programs are selected for execution, and to store instructions and data that are read during program execution. The memory 236 may be volatile or non-volatile and may comprise a read-only memory (ROM), random-access memory (RAM), ternary content-addressable memory (TCAM), dynamic random-access memory (DRAM), and static random-access memory (SRAM).

The memory 236 is operable to store humidity measurements 240, a pre-set humidity threshold, alerts 244, and instructions 238. The instructions 238 may include any suitable set of instructions, logic, rules, or code operable to execute the controller 230.

The network interface 234 is configured to enable wired and/or wireless communications. The network interface 234 is configured to communicate data between the controller 230 and other devices, systems, or domains (e.g., within the dehydrator unit 110 or external to the dehydrator unit 110). For example, the network interface 234 may comprise a Wi-Fi interface, a LAN interface, a WAN interface, a modem, a switch, or a router. The processor 232 is configured to send and receive data using the network interface 234. The network interface 234 may be configured to use any suitable type of communication protocol as would be appreciated by one of ordinary skill in the art. Network interface 234 may be configured to communicate with electronic components and devices within the dehydrator unit 110 or components and devices external to the dehydrator unit 110. For example, the controller 230 may use the network interface 234 with the humidity sensor 208 to receive humidity measurements 240 measured by the humidity sensor 208. The humidity measurements 240 received via the network interface 234 may be stored in the memory 236. In another example, the controller 230 may use the network interface 234 to a computing node (not shown) external to the dehydrator unit 110 to receive the humidity threshold 242, which may be stored in memory 236. For example, as shown in FIG. 2, the dehydrator unit 110 includes an ethernet port 210 that may allow a service technician to connect a computing device to the dehydrator unit 110 and transmit the humidity threshold 242 to the controller 230. In one embodiment, the ethernet port 210 may be part of the network interface 234.

The controller 230 may be configured to regulate moisture inside the network cabinet 102 by controlling humidity inside the network cabinet 102. For example, the controller 230 may be configured to turn on one or more front fans 206, one or more back fans 304 (shown in FIG. 3B), or a combination thereof when the humidity level inside the network cabinet 102 exceeds the pre-set humidity threshold 242. Additionally, or alternatively, the controller 230 may be configured to turn off the one or more front fans 206, one or more back fans 304, or a combination thereof when the humidity level inside the network cabinet 102 drops to a value equal to or lower than the humidity threshold 242.

Referring back to FIG. 2, the controller 230 may be configured to receive humidity measurements 240 from the humidity sensor 208 based on a pre-configured schedule (e.g., periodically). The controller 230 may be configured to compare each humidity measurement 240 received from the humidity sensor 208 to the pre-set humidity threshold 242. When a particular humidity value is determined to exceed the humidity threshold 242, the controller 230 turns on one or more front fans 206, one or more back fans 304 (shown in FIG. 3B), or a combination thereof. In one embodiment, to account for measurement errors, the controller 230 may be configured to turn on the front fans 206 and/or back fans 304 in response to detecting that a plurality of humidity values measured by the humidity sensor 208 exceed the humidity threshold 242. Turning on the front fans 206 and/or back fans 304 allows air flow across the desiccant cartridges 310 (shown in FIG. 3A), which in turn allows the desiccant cartridges 310 to remove moisture from the air flowing across the desiccant cartridges 310. As more and more moisture is removed by the desiccant cartridges 310 from the air inside the network cabinet 102, the humidity level inside the network cabinet 102 may start to drop and may eventually drop below the humidity threshold 242. Upon detecting that one or more humidity values measured by the humidity sensor 208 equal or are lower than the humidity threshold 242, the controller 230 may be configured to turn off the one or more front fans 206, the one or more back fans 304, or the combination thereof that were previously turned on. In one embodiment, to reduce the time taken to lower the humidity levels inside the network cabinet 102, the controller 230 may turn on all front fans 206 and all back fans 304 to maximize the air flow across the desiccant cartridges 310 and cause faster removal of moisture from air inside the network cabinet 102.

In some cases, as the dehydrator unit 110 is used to remove moisture from the network cabinet 102, over time the desiccant cartridges 310 reach their maximum moisture saturation or near maximum moisture saturation, thus loosing their ability to effectively remove moisture from the air. At this time, to continue regulating moisture inside the network cabinet 102 effectively and efficient, the desiccant cartridges need to be replaced or regenerated/reactivated using heat to dry out the moisture. The controller 230 may be configured to detect when a desiccant cartridge 310 has reached its maximum moisture saturation or near maximum saturation and has lost its ability to effectively remove moisture from air. The controller 230 may be configured to determine that the humidity level inside the network cabinet 102 has not dropped below the humidity threshold 242 after running the front fans 206 and/or back fans 304 for a pre-configured time period. In response to this determination, the controller 230 may determine that the one or more desiccant cartridges 310 have reached maximum moisture saturation or near maximum saturation and generate and transmit an alert indicating that the one or more desiccant cartridges 310 have reached maximum moisture saturation or near maximum saturation.

For example, after turning on one or more front fans 206 and/or one or more back fans 304, the controller continues to monitor humidity measurements 240 made by the humidity sensor 208. When, after a pre-configured time period has passed since turning on the front fans 206 and/or back fans 304, the controller 230 determines that one or more humidity values measured by the humidity sensor 208 have not dropped to be equal or lower than the humidity threshold 242, the controller 230 generates and transmits an alert that includes one or more latest humidity values measured by the humidity sensor. The alert servers as an indication that the one or more desiccant cartridges 310 have reached maximum moisture saturation or near maximum saturation. In response to receiving the alert (e.g., at a control center), a service technician may be dispatched to replace the desiccant cartridges 310 or regenerate the desiccant cartridges 310 using heat to dry out the moisture.

FIG. 4 is a flowchart of an example method 400 for regulating moisture in a network cabinet 102, in accordance with embodiments of the present disclosure. Method 400 may be performed by the controller 230 as shown in FIG. 2 and described above.

At operation 402, the controller 230 receives a humidity value measured by the humidity sensor 208 of the dehydrator unit 110.

As described above, the controller 230 may be configured to regulate moisture inside the network cabinet 102 by controlling humidity inside the network cabinet 102. Referring back to FIG. 2, the controller 230 may be configured to receive humidity measurements 240 from the humidity sensor 208 based on a pre-configured schedule (e.g., periodically).

At operation, 404, the controller 230 compares the measured humidity value to the pre-set humidity threshold 242.

At operation 406, based on comparing the measured humidity value to the pre-set humidity threshold 242, the controller 230 determines whether the humidity value received from the humidity sensor 208 exceeds the humidity threshold 242. If the humidity value is found to be lower than or equal the humidity threshold 242, the controller 230 continues to receive humidity measurements 240 from the humidity sensor 208 at operation 402. On the other hand, if humidity value received from the humidity sensor 208 exceeds the humidity threshold 242, method 400 proceeds to operation 408.

At operation 408, in response to determining that the first humidity value exceeds the pre-set humidity threshold 242, the controller 230 turns on the one or more front fans 206, the one or more back fans 304, or a combination thereof.

As described above, the controller 230 may be configured to compare each humidity measurement 240 received from the humidity sensor 208 to the pre-set humidity threshold 242. When a particular humidity value is determined to exceed the humidity threshold 242, the controller 230 turns on one or more front fans 206, one or more back fans 304 (shown in FIG. 3B), or a combination thereof. In one embodiment, to account for measurement errors, the controller 230 may be configured to turn on the front fans 206 and/or back fans 304 in response to detecting that a plurality of humidity values measured by the humidity sensor 208 exceed the humidity threshold 242. Turning on the front fans 206 and/or back fans 304 allows air flow across the desiccant cartridges 310 (shown in FIG. 3A), which in turn allows the desiccant cartridges 310 to remove moisture from the air flowing across the desiccant cartridges 310. As more and more moisture is removed by the desiccant cartridges 310 from the air inside the network cabinet 102, the humidity level inside the network cabinet 102 may start to drop and may eventually drop below the humidity threshold 242. Upon detecting that one or more humidity values measured by the humidity sensor 208 equal or are lower than the humidity threshold 242, the controller 230 may be configured to turn off the one or more front fans 206, the one or more back fans 304, or the combination thereof that were previously turned on. In one embodiment, to reduce the time taken to lower the humidity levels inside the network cabinet 102, the controller 230 may turn on all front fans 206 and all back fans 304 to maximize the air flow across the desiccant cartridges 310 and cause faster removal of moisture from air inside the network cabinet 102.

In some cases, as the dehydrator unit 110 is used to remove moisture from the network cabinet 102, over time the desiccant cartridges 310 reach their maximum moisture saturation or near maximum moisture saturation, thus loosing their ability to effectively remove moisture from the air. At this time, to continue regulating moisture inside the network cabinet 102 effectively and efficient, the desiccant cartridges need to be replaced or regenerated/reactivated using heat to dry out the moisture. The controller 230 may be configured to detect when a desiccant cartridge 310 has reached its maximum moisture saturation or near maximum saturation and has lost its ability to effectively remove moisture from air. The controller 230 may be configured to determine that the humidity level inside the network cabinet 102 has not dropped below the humidity threshold 242 after running the front fans 206 and/or back fans 304 for a pre-configured time period. In response to this determination, the controller 230 may determine that the one or more desiccant cartridges 310 have reached maximum moisture saturation or near maximum saturation and generate and transmit an alert indicating that the one or more desiccant cartridges 310 have reached maximum moisture saturation or near maximum saturation.

For example, after turning on one or more front fans 206 and/or one or more back fans 304, the controller continues to monitor humidity measurements 240 made by the humidity sensor 208. When, after a pre-configured time period has passed since turning on the front fans 206 and/or back fans 304, the controller 230 determines that one or more humidity values measured by the humidity sensor 208 have not dropped to be equal or lower than the humidity threshold 242, the controller 230 generates and transmits an alert that includes one or more latest humidity values measured by the humidity sensor. The alert servers as an indication that the one or more desiccant cartridges 310 have reached maximum moisture saturation or near maximum saturation. In response to receiving the alert (e.g., at a control center), a service technician may be dispatched to replace the desiccant cartridges 310 or regenerate the desiccant cartridges 310 using heat to dry out the moisture.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

To aid the Patent Office, and any readers of any patent issued on this application in interpreting the claims appended hereto, applicants note that they do not intend any of the appended claims to invoke 35 U.S.C. § 112 (f) as it exists on the date of filing hereof unless the words “means for” or “step for” are explicitly used in the particular claim.

1. A dehydrator unit comprising:
an enclosed housing that houses one or more removable desiccant cartridges, wherein the one or more desiccant cartridges are designed to remove moisture from air;
a front panel disposed at a front end of the enclosed housing, wherein the front panel comprises one or more front fans that causes air to flow into the enclosed housing;
a back panel disposed at a back end of the enclosed housing, wherein the back panel comprises one or more back fans that causes air to flow out of the enclosed housing;
a humidity sensor configured to measure a humidity level in air surrounding the dehydrator unit; and
a controller comprising:
a memory that stores a pre-set humidity threshold; and
a processor communicatively coupled to the memory and the humidity sensor, wherein the processor is configured to:
receive a first humidity value measured by the humidity sensor;
compare the first humidity value to the pre-set humidity threshold;
determine, based on the comparing, that the first humidity value exceeds the pre-set humidity threshold; and
in response to determining that the first humidity value exceeds the pre-set humidity threshold, turn on the one or more front fans, the one or more back fans, or a combination thereof.

2. The dehydrator unit of claim 1, wherein the processor is further configured to:
receive a second humidity value measured by the humidity sensor;
compare the second humidity value to the pre-set humidity threshold;
determine, based on comparing the second humidity value to the pre-set humidity threshold, that the second humidity value equals or is lower than the pre-set humidity threshold; and
in response to determining that the second humidity value equals or is lower than the pre-set humidity threshold, turn off the one or more front fans, the one or more back fans, or the combination thereof.

3. The dehydrator unit of claim 1, wherein the processor is further configured to:
after turning on the one or more front fans, the one or more back fans, or the combination thereof:
monitor humidity values measured by the humidity sensor according to a pre-configured schedule;
determine, based on monitoring the humidity values, that the humidity in the air surrounding the dehydrator unit has not dropped to equal or lower than the humidity threshold within a pre-configured time period from turning on the one or more front fans, the one or more back fans, or the combination thereof; and
in response to determining that the humidity in the air surrounding the dehydrator unit has not dropped to equal or lower than the humidity threshold within a pre-configured time period from turning on the one or more front fans, the one or more back fans or the combination thereof, transmit an alert that comprises a latest humidity value measured by the humidity sensor.

4. The dehydrator unit of claim 1, wherein:
the front panel comprises a front fan array having a plurality of the front fans that cause the air to flow into the enclosed housing; and
the back panel comprises a back fan array having a plurality of the back fans that cause the air to flow out of the enclosed housing.

5. The dehydrator unit of claim 1, further comprising:
a network interface configured to communicate with one or more computing nodes; and
wherein the processor is further configured to:
receive the humidity threshold via the network interface; and
store the received humidity threshold in the memory.

6. The dehydrator unit of claim 1, further comprising:
a desiccant tray configured to be housed inside the enclosed housing and hold the one or more removable desiccant cartridges on a top surface of the desiccant tray, wherein the desiccant tray slides in and out of the enclosed housing; and
one or more indentations provided at a front end of the desiccant tray, wherein each of the one or more indentation is configured to receive a finger of a user for holding the desiccant tray and sliding out the desiccant tray from the enclosed housing.

7. The dehydrator unit of claim 6, wherein the front panel is configured to be opened to expose the front end of the desiccant tray.

8. The dehydrator unit of claim 6, wherein at least the front end of the desiccant tray facing the front panel and a back end of the desiccant tray facing the back panel is made of a mesh material to allow air flow across desiccant tray.

9. The dehydrator unit of claim 1, wherein the dehydrator unit is configured to be deployed inside an enclosed cabinet designed to house electronic equipment.

10. The dehydrator unit of claim 1, wherein each of the one or more desiccant cartridges comprises a desiccant material that removes moisture from ambient air.

11. The dehydrator unit of claim 10, wherein the desiccant material changes color to indicate moisture saturation level of the desiccant material.

12. The dehydrator unit of claim 10, wherein the desiccant material comprises silica gel that that changes from a first color when moisture saturation of the silica gel is below a first saturation level to a second color when the moisture saturation of the silica gel exceeds a second saturation level.

13. A method for regulating humidity comprising:
receiving a first humidity value measured by a humidity sensor provided in a dehydrator unit, wherein the dehydrator unit comprises:
an enclosed housing that houses one or more removable desiccant cartridges, wherein the one or more desiccant cartridges are designed to remove moisture from air;
a front panel disposed at a front end of the enclosed housing, wherein the front panel comprises one or more front fans that causes air to flow into the enclosed housing; and
a back panel disposed at a back end of the enclosed housing, wherein the back panel comprises one or more back fans that causes air to flow out of the enclosed housing;
wherein the humidity sensor is configured to measure a humidity level in air surrounding the dehydrator unit;
comparing the first humidity value to a pre-set humidity threshold;
determining, based on the comparing, that the first humidity value exceeds the pre-set humidity threshold; and
in response to determining that the first humidity value exceeds the pre-set humidity threshold, turning on the one or more front fans, the one or more back fans, or a combination thereof.

14. The method of claim 13, further comprising:
receiving a second humidity value measured by the humidity sensor;
comparing the second humidity value to the pre-set humidity threshold;
determining, based on comparing the second humidity value to the pre-set humidity threshold, that the second humidity value equals or is lower than the pre-set humidity threshold; and
in response to determining that the second humidity value equals or is lower than the pre-set humidity threshold, turning off the one or more front fans, the one or more back fans, or the combination thereof.

15. The method of claim 13, further comprising:
after turning on the one or more front fans, the one or more back fans, or the combination thereof:
monitoring humidity values measured by the humidity sensor according to a pre-configured schedule;
determining, based on monitoring the humidity values, that the humidity in the air surrounding the dehydrator unit has not dropped to equal or lower than the humidity threshold within a pre-configured time period from turning on the one or more front fans, the one or more back fans, or the combination thereof; and
in response to determining that the humidity in the air surrounding the dehydrator unit has not dropped to equal or lower than the humidity threshold within a pre-configured time period from turning on the one or more front fans, the one or more back fans, or the combination thereof, transmitting an alert that comprises a latest humidity value measured by the humidity sensor.

16. The method of claim 13, further comprising:
receiving the humidity threshold via a network interface of the dehydrator unit; and
storing the received humidity threshold in a memory associated with the dehydrator unit.

17. A system comprising:
an enclosed cabinet designed to house electronic equipment; and
a dehydrator unit configured to be deployed inside the enclosed cabinet, wherein the dehydrator unit comprises:
an enclosed housing that houses one or more removable desiccant cartridges, wherein the one or more desiccant cartridges are designed to remove moisture from air;
a front panel disposed at a front end of the enclosed housing, wherein the front panel comprises one or more front fans that cause air to flow into the enclosed housing;
a back panel disposed at a back end of the enclosed housing, wherein the back panel comprises one or more back fans that cause air to flow out of the enclosed housing;
a humidity sensor configured to measure a humidity level in air surrounding the dehydrator unit; and
a controller comprising:
a memory that stores a pre-set humidity threshold; and
a processor communicatively coupled to the memory and the humidity sensor, wherein the processor is configured to:
receive a first humidity value measured by the humidity sensor;
compare the first humidity value to the pre-set humidity threshold;
determine, based on the comparing, that the first humidity value exceeds the pre-set humidity threshold; and
in response to determining that the first humidity value exceeds the pre-set humidity threshold, turn on the one or more front fans, the one or more back fans, or a combination thereof.

18. The system of claim 17, wherein the enclosed cabinet is a network cabinet deployed at a cell site, wherein the network cabinet is configured to store electronic equipment associated with operation of the cell site.

19. The system of claim 17, wherein the processor is further configured to:
receive a second humidity value measured by the humidity sensor;
compare the second humidity value to the pre-set humidity threshold;
determine, based on comparing the second humidity value to the pre-set humidity threshold, that the second humidity value equals or is lower than the pre-set humidity threshold; and
in response to determining that the second humidity value equals or is lower than the pre-set humidity threshold, turn off the one or more front fans, the one or more back fans, or the combination thereof.

20. The system of claim 17, wherein the processor is further configured to:
after turning on the one or more front fans, the one or more back fans, or the combination thereof:
monitor humidity values measured by the humidity sensor according to a pre-configured schedule;
determine, based on monitoring the humidity values, that the humidity in the air surrounding the dehydrator unit has not dropped to equal or lower than the humidity threshold within a pre-configured time period from turning on the one or more front fans, the one or more back fans, or the combination thereof; and
in response to determining that the humidity in the air surrounding the dehydrator unit has not dropped to equal or lower than the humidity threshold within a pre-configured time period from turning on the one or more front fans and the one or more back fans, transmit an alert that comprises a latest humidity value measured by the humidity sensor.
